(19) United States
(12) Patent Application Publication
(10) Pub. No.: US 2012/0018528 A1
(43) Pub. Date: Jan. 26, 2012

(54) METHOD OF DISPENSING AT LEAST ONE TEST SCENT

(75) Inventor: Henri Samain, Bievres (FR)

(73) Assignee: L'OREAL, Paris (FR)

(21) Appl. No.: 13/129,880

(22) PCT Filed: Nov. 24, 2009
(86) PCT No.: PCT/IB2009/055309

§ 371 Date: Sep. 7, 2011

Related U.S. Application Data
(60) Provisional application No. 61/193,478, filed on Dec. 3, 2008.

(30) Foreign Application Priority Data
Nov. 24, 2008 (FR) 08 57958

(51) Int. Cl. A61L 9/04; G06F 15/16

(52) U.S. Cl. 239/6; 709/219

(57) ABSTRACT

A method of dispensing at least one test scent within a system includes a server configured to exchange data with a plurality of interactive scent-formulation terminals, each interactive terminal including a plurality of odorous substances, serving when combined in various proportions to create a plurality of scents; dispenser means for dispensing a selection of the odorous substances out from the terminal; a network interface enabling the terminal to communicate with the server; and a user interface; in which method the server automatically sends data to at least one terminal, the data describing at least one formulation enabling the terminal to dispense a test scent, while leaving the user of the terminal free to select the moment at which the test scent is dispensed.

The present invention relates to preparing odorous compositions, in particular fragrances.

It is known, e.g. from patents EP 0 993 625 and U.S. Pat. No. 6,556,272, and from application DE 100 18 914, to dispense a scent in the environment of a user, the scent being selected relative to a multimedia content being played to the user.

Application FR 2 841 081 discloses encoding data on a multimedia content medium, which data relates to an odor, so that the odor is diffused while the multimedia content is being played back.

Application US 2004/0204043 teaches enabling a user of a mobile telephone to send a message to another mobile telephone describing an odor, with reception of the message leading to the odor being diffused by the mobile telephone. That application US 2004/0204043 also teaches enabling a user of a terminal to download a formulation from an Internet site to enable it to be used.

There exists a need to simplify from the user point of view the reception of formulations enabling test scents to be prepared and enabling the scents to be fabricated.

### SUMMARY OF THE INVENTION

Exemplary embodiments of the invention provide a method of dispensing at least one test scent within a system comprising a server configured to exchange data with a plurality of interactive scent-formulation terminals, each interactive terminal comprising:
a plurality of odorous substances, serving when combined in various proportions, to create a plurality of scents;
dispenser means for dispensing a selection of the odorous substances out from the terminal;
a network interface enabling the terminal to communicate with the server; and
a user interface;

in which method the server automatically sends data to at least one terminal, the data describing at least one formulation enabling the terminal to dispense a test scent, while leaving the user of the terminal free to select the moment at which the test scent is dispensed.

The invention makes it possible to propose dispensing the scent to the user of the terminal without constraining the user of the terminal to select a formulation and/or to adjust for the quantity of scent that is to be diffused. For example, the invention makes it possible to simplify and improve techniques for promoting scents, with the formulations of the scents being sent automatically to the terminals.

The invention may serve to enable a user of the terminal to receive, in any location, a formulation enabling a test scent to be dispensed, and to dispense the corresponding scent.

By way of example the server automatically sends the data describing the same formulation to a plurality of terminals.

The data automatically sent by the server may describe a formulation that has not been previously reproduced by the terminal.

According to the invention, the formulation transmitted by the server to a terminal is reproduced only on instruction of the user of the terminal and not automatically after being received by the terminal.

The term “reproduce a formulation” is used to mean dispensing the scent that corresponds to the formulation in order to enable it to be diffused or packaged.

The formulation may be sent by the server to the terminal independently of the terminal sending a request for a formulation to be returned thereto, e.g. independently of a request for the return of a formulation that is optimized by the server on the basis of a formulation prepared by the user of the first terminal or independently of a request for the return of a formulation as determined by the server on the basis of the list of odorous substances available at the terminal or that may be associated therewith.

By way of example, the formulation that is sent may correspond to a scent that is already on sale or that is about to be put on sale.

In a variant, the formulation that is sent is a simplified formulation for a scent that is on sale or that is about to be put on sale, for the purpose of giving the user an overall impression that closely matches that scent. The term “simplified formulation of a scent” is used to mean a formulation with fewer nuances than said scent, with a smaller number of odorous substances being used to define the simplified formulation, for example.

By way of example, the use of a simplified formulation makes it possible to avoid using too great a number of odorous substances for reproducing the formulation of said scent, thereby avoiding the need for the user to associate too large a number of distinct odorous substances with the terminal, while still giving a perception of said scent that is a relatively good match.

The user of the terminal may select one or more substances for dispensing with the help of the user interface.

By way of example, the formulation that is sent for the test scent, as described in the data sent by the server, is determined as a function of user preferences associated with the user of the terminal to whom the data has been sent, thus making it possible for the scent to better satisfy the desires of the user of the terminal.

The user preferences may include one or more stored formulations, where appropriate.

For example, whether or not the data describing the formulation of the test scent is sent to the terminal may be conditional on the server detecting a predefined condition associated with the user of the terminal, in particular a condition relating to the location of the terminal, to the use of the terminal, to the use of computerized payment means, or to the availability of odorous substances at the user's terminal or capable of being associated therewith.

The condition relating to the user of the terminal may for example be stored in a memory associated with the terminal, with this condition being constituted, for example, by at least some information relating to earlier purchases of scents by the user.

The condition relating to the user of the terminal may for example relate to the age, the sex, the taste, or the purchasing habits of the user, and may also relate to certain particular dates such as celebrations, commemorations, anniversaries or birthdays, or indeed to the user's family and acquaintances, e.g. in a family, social, or professional context.

A scent formulation may be sent with given periodicity to comply with a preference expressed by the user.

The terminal may include a positioning system, e.g. of the global positioning system (GPS) type, or associated with mobile telephone technology, and the condition relating to the location of the terminal may be true when the terminal is close to a scent shop, for example.

In another variant, the above predefined condition relates to the user taking an action on the user interface of the terminal, for example an action causing the terminal to send to the server a request that it return a formulation, thus making it possible to take account of a sudden desire on the part of the user of the terminal to test a scent.

In a variant, the action of the user on the user interface programs the terminal so that it can subsequently receive one or more formulations of test scents, where appropriate providing one or more other predefined conditions are satisfied. By way of example, programming the terminal may consist in sending to the server a request for subsequent acceptance of formulations, where appropriate during a specified period or as a function of criteria relating to formulations, e.g. as a function of olfactive descriptors.

The condition relating to the use of the terminal may for example be at least one condition selected from the following: the frequency with which the user makes use of the terminal; the scents recently dispensed by the terminal; the purchase date of the odorous substances available at the terminal; or indeed the date on which said substances were placed in the terminal.

When the predefined condition relates to the odorous substances available at the terminal, the server may inform the terminal of the substance(s) listed in the formulation that are needed for dispensing the test scent and that are not available at the terminal.

By way of example, the server may interrogate a memory associated with the terminal prior to sending data to the terminal describing the formulation of the test scent in order to determine whether or not the predefined condition(s) is/are satisfied.

The term “memory associated with a terminal” is used to mean a memory that may be incorporated in the terminal or a memory that may be situated outside the terminal and that is accessible from the terminal or that is accessible by the server.

In a variant, the terminal sends a request to the server including data indicating that one or more predefined conditions are satisfied.

The user of the terminal may order a product associated with the test scent of formulation described in the data sent by the server. By way of example, the product may be a packaged product associated with the test scent. The term “packaged product associated with the test scent” designates a bottle or any other packaging containing the scent, and also body care products such as scented soap, shower gel, or indeed shampoo. Such a packaged product may also be a washing powder.

In a variant, the product associated with the scent is a solid article, such as a garment, paper, or indeed envelopes.

The data describing the formulation of the test scent as sent by the server may refer to a formulation stored in a memory associated with the terminal.

The stored formulation may be hidden, with access to said formulation by the user of the terminal being made possible only after receiving authorization delivered by the server.

The server may debit or credit an account associated with the terminal when the user of the terminal orders dispensing of the test scent.

The user of the terminal receiving the data describing the formulation of the test scent may modify the received formulation. By way of example, the modification may be performed by the user inputting at least one olfactive descriptor via the user interface of the terminal.

The server may take account of the modifications made by the user when making a subsequent proposal of a formulation to the terminal, thus making it possible to optimize the creation of scents and/or the dispensing of scents and/or establishing personalized dispensing of scents.

The user of the terminal that has received the data describing the formulation enabling the test scent to be dispensed may forward the data to another terminal selected by the user, with this being accompanied by a message, for example. By way of example, the user may send an opinion about the dispensed test scent.

The server may send multimedia content associated with data describing the formulation enabling the test scent to be dispensed, where such multimedia content may be constituted, for example, by video and/or audio content.

Other exemplary embodiments of the invention also provide a server configured to exchange data with a plurality of interactive scent-formulation terminals, each terminal comprising:
a plurality of odorous substances, serving when combined in various proportions, to create a plurality of scents;
dispenser means for dispensing a selection of the odorous substances out from the terminal;
a user interface; and
a network interface enabling the terminal to communicate with the server, so as to receive data from the server describing at least one formulation enabling a test scent to be dispensed;

the server being configured to send data automatically to at least one terminal, the data describing at least one formulation enabling at least one test scent to be dispensed, and to receive from the terminal a message confirming that the test scent has been dispensed.

The automatic sending of the formulation for the test scent may be independent of any prior transmission of a request from the terminal.

The server may send data to the terminal describing the formulation of the test scent independently of multimedia content, e.g. video or audio content, being played back in the environment of the user of the terminal.

In a variant, the formulation sent to the terminal is accompanied by the playback of multimedia content.

By way of example, the server is configured to detect whether a predefined condition is satisfied as described above and/or to determine at least one formulation enabling a test scent to be fabricated on the basis of user preferences associated with the user of the terminal and/or to send to said terminal data describing at least one formulation enabling the test scent to be dispensed once said condition is satisfied.

By way of example, on detecting that the predefined condition(s) is/are satisfied or on receiving a request from the terminal stating that the predetermined condition(s) is/are satisfied, the server may be configured to interrogate said terminal prior to sending thereto data describing the formulation enabling the test scent to be dispensed, in order to determine whether the terminal has available the odorous substances listed in the formulation, thus making it possible to avoid the terminal seeking to reproduce a formulation even though it is missing certain odorous substances.

By way of example, the server is configured to determine whether at least one of the odorous substances that are listed in the formulation enabling the test scent to be dispensed and that is/are unavailable at said terminal can be replaced by at least one other odorous substance available at said terminal, in order to obtain a result that is close, e.g. that will be perceived by a nose with little training as being the same scent.

The server may also be configured to transmit to said terminal a request inviting the user of the terminal to place in said terminal at least one odorous substance that is not available at the terminal, in order to reproduce the formulation enabling the test scent to be dispensed.

Other exemplary embodiments of the invention also provide an interactive scent-formulation terminal, comprising:
a plurality of odorous substances enabling scents to be made up;
means for recognizing the substances present at the terminal;
dispenser means enabling a selection of said odorous substances to be dispensed;
a user interface enabling user preferences to be stored in a memory associated with the terminal; and
a network interface enabling data to be received from a server, the data describing at least one formulation enabling the test scent to be dispensed.

The terminal and/or server may for example be arranged to access a correspondence table of odorous substances in order to substitute an odorous substance that is not available at the terminal with at least one other odorous substance that is available thereat.

The dispenser means, the user interface and the network interface may be incorporated in a single device, being for example located inside a same housing.

The odorous substances may be contained in reservoirs that may be placed in the terminal independently of one another. The odorous substances may in particular not be carried by a same sheet.

Terminal

The terminal may be made in the form of a single appliance, however it would not go beyond the ambit of the present invention for the terminal to be made with the help of a plurality of appliances placed in a common location.

By way of example, the terminal may be small in size, e.g. being a portable appliance, suitable for being contained in a handbag, where appropriate. The terminal may be considered as constituting an association of a computer, whether portable or fixed, or of a game console, portable or otherwise, with an internal extension, e.g. a card, an integral external extension, or a non-integral external extension.

By way of example, the terminal includes an independent source of electricity incorporated therein, such as an optionally rechargeable battery.

By way of example, the terminal includes a plurality of reservoirs, e.g. more than two, three, five, or ten reservoirs receiving the substances for dispensing one or more scents. By way of example, the reservoirs are removably mounted in the terminal so as to be capable of being placed during and/or after use of the terminal. The reservoirs are advantageously arranged in such a way that the substances they contain may be exchanged, replaced, or refilled.

The term “substance” is used to designate either a pure substance, or a diluted substance or a mixture of substances whether diluted or not.

A substance may contain 10 to 50, and in particular 10 to 20 compounds. By way of example, the substances are themselves portable and/or used for creating scents.

By way of example, the terminal includes a user interface and a network interface. By way of example, the user interface comprises at least one interface selected from: a keypad, a joystick, a touch pad, a screen, optionally a touch screen, and a voice recognition system.

The terminal also includes dispenser means, as described below, for dispensing the substances contained in the reservoirs, and in selected proportions.

By way of example, the terminal is associated with a memory that may be incorporated in the terminal or that may be external to the terminal and accessible thereby. Amongst other possibilities, the memory may comprise a hard disk, electronic circuits, e.g. of the flash memory type, or an optical disk reader/writer.

The memory associated with the terminal may include scent formulations that have previously been sent by the server to the terminal or by at least one second terminal to said terminal, and/or scent formulations sent by the terminal to the server or to a second terminal.

The memory associated with the terminal may also include user preferences, e.g. as defined by the user of the terminal and as described below. The terminal may also include other modules, as described below.

The terminal may also be configured to enable applications to be provided other than dispensing scents, for example it may be used for applying makeup.

The terminal may include dispenser means dedicated to applying scents to makeup.

### Server

The term “server” designates one or more programmed computers, e.g. capable of managing data exchanges with a large number of terminals.

By way of example, a database is associated with the server, said database being incorporated in the server or being external to the server and accessible therefrom, for example.

The database may list examples of predefined formulations, and/or it may include association rules governing the possibilities and/or the pertinence of associating substances within various formulations.

The database may also include user preferences associated with the users of the terminals.

By way of example, the server may be entirely automatic, i.e. requests coming from a terminal are analyzed solely without human intervention. The formulations corresponding to the scents for testing may also be sent in a manner that is entirely automatic.

In a variant, the server may enable a specialist to intervene in devising scent compositions, where such a person is referred to below as an “expert”.

By way of example, the server may be configured:
to send data automatically to at least one terminal, the data describing at least one formulation enabling at least one test scent to be dispensed, independently of any prior transmission of a request by the terminal; and
to receive a message from the terminal, the message confirming that the test scent has been dispensed.

Receiving a confirmation message may be useful, for example for the purpose of statistical processing of the impact of a promotional campaign, or in order, subsequently, to personalize processing for the user of the terminal.

The server may also be configured:
to receive a request from the terminal, the request including information specifying whether one or more predefined conditions are, or are not, satisfied, and information concerning the substances available at said terminal; and
to analyze the request in order to determine at least one formulation for reproduction.

In a variant, the server is configured to detect that one or more predefined conditions are satisfied by said terminal or the user of the terminal.

For example, when analyzing a request, the server may be configured to access a memory or a database having user preferences associated with the user of the terminal stored therein, and to determine the formulation that is to be reproduced as a function of those user preferences. Such user preferences are described in greater detail below.

On analyzing a request, the server may determine at least one formulation that can be reproduced by the terminal by taking account only of those substances that are available at the terminal.

As described below, the server may be configured to access a correspondence table between odorous substances, for example.

The server may also determine at least one formulation that takes account of at least one other substance that is not available at the terminal but that can be added to the substances that are available to the terminal.

For example, the server may be configured to send a warning message to the terminal when one or more of the substances listed in the formulation determined by the server are not available.

When the terminal includes a source of electricity, the server may also, on analyzing the request, interrogate the terminal concerning the extent to which the independent electricity source is depleted.

After the terminal has dispensed the test scent, the server may also be arranged to debit or credit an account associated with the user of the terminal. By way of example, the account may correspond to a loyalty card enabling users to be recompensed by awarding them points.

The invention is not limited to a single server.

In a variant, the invention implements servers that communicate with one another and that are dedicated to different applications. For example, one server may be dedicated to dispensing scents, and another may be dedicated to preparing makeup and care compositions, which may for example include the generated or optimized formulation as scent.

The various servers may communicate with a terminal capable of taking account of a plurality of the above-described applications or with terminals that are dedicated to one only of those applications.

In another variant, a plurality of servers are dedicated to the same application, e.g. to dispensing scents.

### Formulation Communications Standard

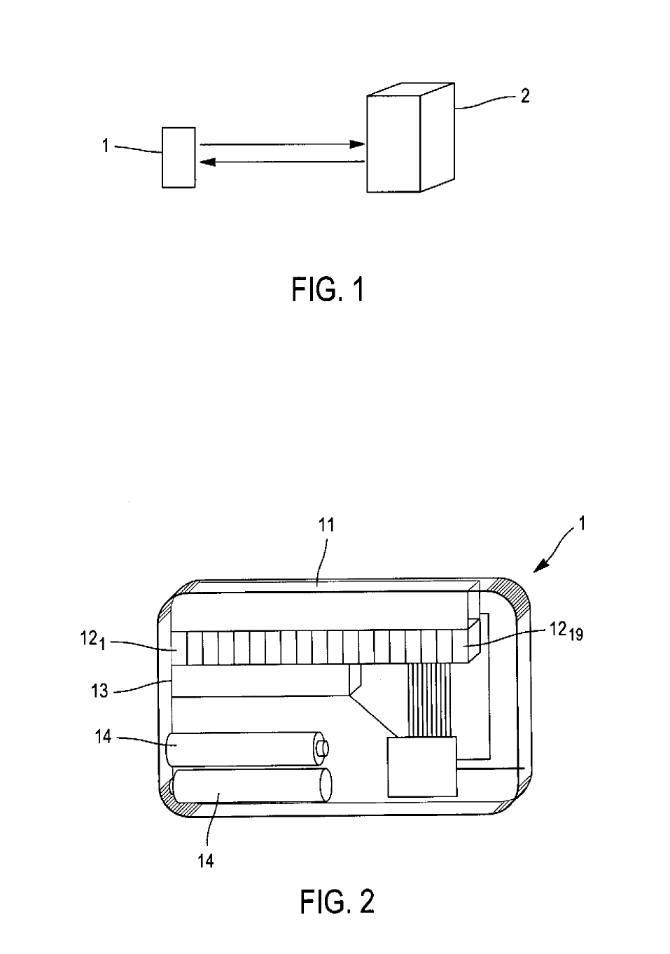

As can be seen in FIG. 1, which is a highly diagrammatic representation of a terminal 1 and a server 2, the invention involves establishing communication between the terminal 1 and the server 2.

This communication may take place using a communications standard, for example.

As described below, the server and each terminal are connected or connectable via a wired or a wireless connection.

By way of example, the communications standard comprises a data transmission protocol enabling substances to be identified and/or describing the content of each substance used in the formulation.

By way of example, the data serving to identify a substance need not include any data describing the properties of the substance, for example it may comprise a code.

The server and/or each terminal may, for example, be arranged to convert the code into information concerning the substance.

In a first example of a standard, the data enabling the substance to be identified comprises no more than information about the location of the reservoir containing the substance in each terminal.

In this first example, the reservoirs containing the substance are disposed in predetermined manner in each terminal, for example.

In a second example of a standard, the data enabling a substance to be identified comprises information for identifying a group of substances, e.g. a palette of substances, together with data enabling the location of the substance within said group of substances to be identified.

In another example of a standard, the information enables each substance to be identified independently of the server of the reservoir that contains it in the terminal.

The data concerning the content of each substance in the formulation is preferably expressed as the total quantities of the substances in the formulation.

These quantities may be expressed in terms of volume or weight, in a metric or other standard, or using a proprietary standard.

In a variant, the information relating to the content of a substance in the formulation comprise at least:
the duration of substance transfer or flow from the reservoir containing the substance to the dispenser means;
the strength and/or the pressure to be applied to the reservoir containing the substance; and/or
the voltage to be applied to an electrical actuator for dispensing the substance from the reservoir.

For at least one of the substances in the formulation, and in particular for each of the substances in the formulation, the standard may also include at least one of the items of information taken from the following list: a required heating temperature; the physical state of the substance, such as its viscosity or color, for example; information concerning incompatibilities when crossed with other substances; a use-by date or a fabrication date or an expiry date for the substance; batch numbers for reasons of traceability; or indeed possible risks associated with a substance, such as allergy risks.

By way of example, the standard may serve to code all of the above-mentioned information, without it being necessary for all of that information always to be contained in the data that is transmitted in a communication.

By way of example, the transmitted data may include information relating to the resulting scent, that is independent of any particular substance, for example it may include at least one of the information items selected from the following list: a heating temperature or some other stimulation; a speed of stirring or stirring conditions, or a time the scent should be allowed to settle; instructions to add substances when reproducing the formulation; information concerning the physical state of the scent, such as its viscosity, its color, or its stability, for example; a use-by date; a fabrication date; or an expiry date of the scent; and possible risks, e.g. allergy risks.

The data transmitted using the communications protocol may also include, for example, information items relating to the purpose of the scent of the formulation, e.g. whether the scent of the formulation is to be introduced into a solvent or into some other substance such as for example: a cosmetic, a foodstuff, a fuel, or a fluid for cleaning or washing linen, dishes, or the floor. Such data may also specify portions of the body on which the scent is to be applied, in particular the neck, the head, the armpits, the feet, or certain articles such as garments, shoes, furniture, or indeed cars.

The transmitted data may also include information for determining whether the scent of the formulation is to be dispensed using a dispenser or a diffuser that is passive or active, e.g. electrical, thermal, or in the form of a spray.

The data may also specify whether the formulation for reproducing is intended for a man, a woman, or a child, or indeed whether the scent of the formulation is intended for a particular age range.

The communications standard may provide information specifying whether the scent of the formulation is for use by groups such as those that can be defined by a common liking of consumer goods, artistic products, actors, singers, or celebrities, or indeed whether the scent of the formulation is for a given person, a given group of people, or a family.

The communications standard may also include information identifying the substances contained in the reservoirs of the first terminal, such as the quantities remaining in the reservoirs.

The transmitted data may also identify the substances present in the reservoirs of a second terminal with which the terminal is connected or with which it has already exchanged information concerning preparing a scent. Where appropriate, the information may also include the remaining quantities of the substances in the reservoirs of the second terminal.

The transmitted data may relate to the context in which the user of the terminal is to be found, such as for example: ambient temperature; ambient humidity; ambient odor; and time of day, taking account of a time shift, where appropriate.

The transmitted data may also relate to the operation of the terminal. For example, such data may be obtained by an automatic self-test performed every time the terminal is to respond or on a periodic basis. This information may relate for example to the speed of fluid flow within the terminal, or to the functioning and/or malfunctioning of certain modules of the terminal.

The communications standard may also provide information concerning the process of communication, grouped together in a header or occupying a plurality of portions. By way of example, this information includes information relating to the identity of the terminal and/or of a user of the terminal, the identity of the server, the identity of a second terminal, identifying the date and/or the communication, identifying a working session, on the terminal or on the server, and/or relating to an encryption system for ensuring that the information is confidential.

As mentioned above, the identities of the substances and the remaining quantities thereof can be represented by distinct parameters. The header may include information specifying which parameters are for use in identifying the substances and/or in quantifying the substance(s) in the formulation, with such information enabling the terminal and/or the server to interpret the data transmitted during a communication.

As explained below, each terminal and the server may be arranged, for example, to operate in a plurality of reproduction modes that differ from one another by the quantity of scent dispensed, and the transmitted data may include, for example, information enabling a reproduction mode to be identified and consequently enabling the quantity of scent that is to be dispensed.

Where appropriate, a plurality of communications standards may be used, and each terminal and/or the server may include a module for converting from one standard to another.

### Substances

As mentioned above, each terminal advantageously includes a plurality of reservoirs receiving substances for dispensing a scent. The terminal includes for example several tens of reservoirs. Each reservoir may contain a different substance. In a variant, a single substance may be present in different reservoirs of the same apparatus, if need be, with different concentrations.

The server may be associated with a plurality of reservoirs.

The substances may be present in a fluid state, e.g. liquid, in each of the reservoirs.

In a variant, at least one portion of the reservoirs includes a substrate on which one or more substances are adsorbed.

All the contained substances may be odorous.

For an odorous substance, compounds or aromas of natural origin or of synthetic origin, and mixtures thereof may be used.

For compounds or aromas of natural origin, mention may be made of flower extracts, e.g. (lilies, lavender, rose, jasmine, ylang-ylang, stalks and leaves (patchouli, geranium, petitgrain), fruit (coriander, anise, cumin, juniper), fruit peel (bergamot, lemon, orange), roots (angelica, celery, cardamom, orris, sweet-flag), wood (pine wood, sandal-wood, pockwood, rose cedarwood), herbs and grasses (tarragon, lemon grass, sage, thyme), needles and branches (spruce, fir, pine, mini pine), resins and balsams (galbanum, elemi gum, benzoin, myrrh, olibanum, opopanax).

For compounds of synthetic origin, mention may be made of ester, ether, aldehyde, ketone, aromatic alcohol and hydrocarbon type compounds, and mixtures thereof.

For esters, particular mention may be made of benzyl acetate, benzyl benzoate, phenoxyethyl isobutyrate, p-tert-butylcyclohexyl acetate, citronellyl acetate, citronellyl formate, geranyl acetate, linalyl acetate, dimethyl-benzylcarbinyl acetate, phenylethyl acetate, linalyl benzoate, benzyl formate, ethylmethylphenyl glycinate, alkylcyclohexyl propionate, styrallyl propionate, and benzyl salicylate.

For ethers, mention may be made of benzylethylether.

For aldehydes, mention may be made of linear alkanals including 8 to 18 carbon atoms, e.g. citral, citronellal, citronellyloxyacetaldehyde, cyclamenaldehyde, hydroxycitronellal, lilial, and bourgeonal.

For ketones, mention may be made of ionones e.g. alpha-isomethylionone, and methylcedrylcetone.

Among aromatic alcohols and terpenics in particular, mention may be made of anethole, citronellol, eugenol, isoeugenol, geraniol, linalol, phenylethyl alcohol, and terpineol.

For hydrocarbons, particular mention may be made of terpenes.

In addition, use may also be made of essential oils, ingredients of an aroma, e.g. essences of sage, camomile, clove, balm, mint, cinnamon tree leaves, linden flowers, juniper, vetiver, olibanum galbanum, labolanum, and lavandin.

By way of example for scent use is made of, on its own or in a mixture, bergamot essence, dihydromyrcenol, lilial, lyral, citronellol, phenylethyl alcohol, alpha-hexylcinnamaldehyde, geraniol, benzylacetone, cyclamenaldehyde, linalol, ambroxan, indole, hedione, sandelice, essences of lemon, mandarin and orange, allylamine glycolate, cyclovertal, lavandin essence, sage essence, betadamascone, geranium essence, cyclohexyl salicylate phenylacetic acid, geranyl acetate, benzyl acetate, and rose oxide,

In a variant, a portion of the substances contained is not odorous, e.g. a reservoir may contain a non-odorous substance.

Still in a variant, one or more reservoirs may contain a substance designed to become odorous after a physical transformation.

By way of example, certain reservoirs may contain substances that are additives such as coloring agents, preservatives, or rheology agents.

FIG. 2 shows an example of terminal 1 of the invention.

As can be seen, terminal 1 may include two types of reservoir: at least one main reservoir 11 and a plurality of nuance reservoirs 12ᵢ.

In the embodiment described and shown, the terminal 1 includes only one main reservoir 11 and nineteen nuance reservoirs 12₁ to 12₁₉.

The term “main reservoir” is used for a reservoir containing sophisticated combinations of odorous substances, e.g. more than five, ten, or fifteen. Such combinations can be considered to be scents, or simple scent compositions. The contents of a main reservoir is for example in the range 1 milliliter (mL) to 50 mL, for example equal to 10 mL

### Storing Substances and Formulations in Memory

One or more memories, here referred to generally as “memory”, may be associated with each terminal and/or server.

The memory may be of any known type, e.g. electronic, optical, or electromagnetic.

The memory may be permanent or erasable and may have protected access.

The memory may be permanently fastened to each terminal. In a variant, the memory is removable from the terminal.

Generally, the terminal may include one to ten main reservoirs for example, in particular one to four, and ten to forty nuance reservoirs.

The nuance reservoirs may contain at least one substance that is predominantly:
woody, e.g. derived from sandal-wood, cedar wood, or vetiver;
citrus, such as lemon essential oil, bergamot essential oil, or essential oil from orange flowers (neroli)
musky, such as white musk, galaxolide;
marine, such as calone;
gourmand, e.g. ethylmaltol or ethylvanilline.
floral, e.g. jasmine absolute or lilial absolute;
oriental, such as patchoulol;
citrus, such as lemon essential oil, bergamot or neroli); and
fruity, such as lagamma-decalactone. As can be seen, terminal 1 may further include a reservoir 13 containing a liquid for rinsing, which is described below.

In FIG. 2 an independent electrical energy source of terminal 1 is also shown, in the embodiment described said energy source is two batteries 14.

At least one reservoir can be divided into a plurality of sub-portions.

By way of example the substances are fluid, in particular they are liquids of low viscosity.

At least one reservoir may receive the composition below (by weight):
5 to 50% odorous substance;
0 to 5% preservative substances;
0 to 20% rheological substances;
0 to 2% coloring agents; and
23 to 95% solvent

By way of example the solvent(s) is/are selected from water, ethanol, acetone, and other polar organic solvents.

At least one of the substances may also be selected from those substances recommended for the treatment of pathologies, infectious or otherwise, such as respiratory disorders e.g. asthma, or skin disorders.

By way of example, at least one of the substances is selected from those substances that, while also being odorous, are recommended for the treatment of a pathology or as relaxants or stimulants. Such substances may be natural substances, e.g. based on natural essences or essential oils, e.g. thyme, frankincense, or even eucalyptus.

### User Interface

The user interface of each terminal includes, for example, a screen on which a formulation is displayed that enables a test scent to be dispensed as received by the terminal from the server.

Each terminal may additionally or as a variant include a printer.

By way of example, the user interface includes one or more keys enabling the user to select a formulation that has been received after being transmitted by the server once the predefined condition(s) apply(ies).

The user interface may also be configured to enable the user to input information relating to user preferences.

The server may also include a user interface as described above. For example, the user interface may enable the expert associated with the server to devise a formulation.

In a variant, the user interface of the terminal, and where appropriate of the server, is configured so as to enable the user and/or the expert to input one or more olfactive descriptors associated with the scent for dispensing, said descriptor(s) possibly being converted with the help of a conversion table into substances so as to lead to at least one formulation.

The conversion table may be contained in the memory associated with the terminal.

In a variant, the conversion table is contained in the database associated with the server.

By using the conversion table, the olfactive descriptors input by the user and/or the expert by means of the user interface may be translated into substances.

The conversion table may also be programmed by the user and/or the expert. The personal experience of the user and/or of the expert may enable certain odor nuances to be associated with olfactive descriptors, and the descriptors may be added to the conversion table, after being previously defined, thereby enabling the conversion table to be personalized.

The personalized conversion table may be transmitted by the terminal to the server and the formulation enabling the test scent to be dispensed may be transmitted by the server, e.g. when the predefined condition applies and may be expressed as a function of this personalized conversion table.

The user interface may enable the user of the terminal to send a formulation accompanied by a message, or where appropriate, to send only a message. The message may include an explanation for the refusal by the user of the terminal to reproduce the formulation sent by the server, or an assessment of the received formulation.

The user interface may also be configured to enable the user of the terminal that has received the formulation enabling the test scent to be prepared, to modify the formulation, where appropriate.

By way of example, the user interface may be configured to propose one or more of the following actions to the user:
to increase or limit the head, heart, or base notes of the scent;
to act on parameters commonly used by perfumers,
such as: citrous, musk, woody, floral nuances;
to act on parameters used in public language such as the notions of “green”, “heavy”, “light”;
to act on the parameters used for food, such as “sweet”, “peppery”, . . . ;
to act on odors that are to be found in nature such as those of flowers, trees, earth, . . . ;
to act on the odors of famous scents or the odors of odorous substances, for example.

The terminal may include artificial intelligence modules for assisting in determining the formulation(s) and/or for preventing or recommending avoidance of certain formulations or modifications to formulations.

The user interface of the terminal may be configured in such a manner that the terminal is capable of acting in slave mode to reproduce received formulations at a single click.

The user interface may enable the user of the terminal to order a product associated with the scent dispensed in accordance with the formulation enabling the test scent to be dispensed, such as, for example, a packaged product such as a bottle containing the scent, but also body care products such as scented soaps, shower gel, or indeed shampoo or a washing powder, or even solid articles such as garments.

By way of example, the terminal may be configured to execute a program causing the following to be displayed on the screen of the user interface:
a number of points corresponding to the number of main reservoirs of the terminal; and
a number of vectors corresponding to the number of nuance reservoirs of the terminal starting from each point displayed on the screen.

Such a user interface may provide a clear representation of the possibilities of formulation from the substances available at the terminal.

The memory may include information relating to the substances present in the reservoirs of each terminal and/or to the remaining quantities of said substances in the reservoir.

The formulations recorded in the memory associated with a terminal may also include hidden formulations, stored in the memory during fabrication of the terminal and accessible only with the help of a code, the code being transmitted for example by the server to the terminal.

The recorded information may also include dates for reproducing previously-received formulations or for using previously-reproduced formulations, together with commentaries received from the server relating to said formulations, such as assessments, for example.

The information stored in the memory associated with the terminal may include, for example, a list of the substances available in the reservoirs of at least one second terminal.

User preferences may also be stored in the memory.

Such user preferences comprise, for example, earlier choices made by the user of the terminal, scent mixing logic, and optionally fashion trends in scent matters, as selected by the user. User preferences may also include information relating to the context in which the user of the terminal is to be found, such as for example: ambient temperature, ambient humidity, ambient odor, time of day.

User preferences may also include scent formulations previously sent by the server to the terminal or by at least one second terminal to said terminal, and/or scent formulation sent by the terminal to the server or to a second terminal.

User preferences may also comprise formulations for scents previously purchased by the user of the terminal.

User preferences may also include information specifying whether the formulation to be reproduced is for a man, a woman, or a child, or indeed whether the scent of the formulation is for a particular age group, and also information specifying whether the scent of the formulation is for groups such as those that can be defined by a common liking for consumer goods, artistic products, actors, singers or celebrities, or indeed whether the scent of the formulation is for a given person, a given group of people, or a family.

User preferences may also define the periodicity with which the user desires to receive formulations for dispensing said scents.

A given terminal may be associated with different users, and the memory associated with the terminal may be divided into sub-memories, each sub-memory being associated with one particular user and containing the formulations reproduced and/or devised by that user.

A single memory may be associated with a plurality of terminals and that memory may for example be divided into units, with each unit being associated with one terminal and containing the above-mentioned information.

Where appropriate, a terminal amongst a plurality of terminals associated with the memory is used by different users, and the unit in question is divided into sub-units, like the above-described sub-memories.

The database associated with the server may store formulations previously sent by the server to the terminal or to at least one second terminal, and/or formulations sent by said terminal or a second terminal to the server. Where appropriate, the assessments sent by the terminal with the formulations may also be stored in the database.

The database may also store the substances that are available at the terminal, together with the remaining quantities thereof.

The database may also store the dates at which substances were placed in the terminal by the user of the terminal.

The information relating to the substances available at the terminal may be updated on receiving information input by the user via the user interface of the terminal or indeed from a substance dispatching center.

The database may also include a correspondence table between odorous substances, as described below.

At least part of the memory associated with the terminal and/or the database may constitute the subject matter of commercial exchanges.

During such exchanges, the portion of the memory and/or the database that is exchanged may be accompanied by other components such as decryption keys, user manuals, a protective housing, odorous samples, suggestions for use or for accompanying products, accompanying products such as those for receiving the formulation, e.g. a dispenser system, products, garments, . . . .

The memory and/or the database may be addressable, for example.

Where appropriate, the user of the terminal and/or the server may access the formulations stored in the memory associated with a second terminal.

Where appropriate, the user of the terminal and/or the server may access information recorded in a database associated with other servers.

### Identifying Available Substances

In an implementation of the invention, a terminal has information relating only to the locations of reservoirs within the terminal. If the reservoirs are not properly positioned, the final odor will not be the expected odor.

In another implementation of the invention, the terminal includes a system for identifying the available reservoir.

The identification system may be of any type and it may include an identifier device and an identifier.

The identifier device may for example be secured to the terminal.

The identifier may be secured to the packaging device containing the substance and may optionally be placed inside the reservoir.

The identification system may for example implement:
radio waves, and in particular radiofrequency identity (RFID) chips;
optical identification such as bar codes or colors;
electrical or electronic connectors; and/or
portions in relief.

By way of example, the identifier is placed on or under or on the side of the reservoir containing the substance.

The terminal may include as many identifier devices as there are reservoirs. In a variant, the number of identifier devices is smaller than the number of reservoirs.

In another variant, the terminal has only one identifier device in common for all of the reservoirs.

When the identification system implements RFID chips, the identifier device may have only one transmitter antenna that addresses the identifiers in the form of RFID chips sequentially.

When an optical identification system is used, the identifier device may comprise a single movable reader capable of being moved to read the identifier associated with each of the reservoirs.

In another variant, the identifier device is stationary relative to the terminal and each terminal is arranged to move the reservoir so that the identifier of said substance is read by the identifier device before the substance is put into place in order to refill a selected reservoir. In a variant, the user presents a substance to the identifier device so as to enable the identifier device to identify the substance, and then the user fills the reservoir with the substance. Each terminal may thus be informed manually or automatically about the location of the reservoir in which the substance has been placed.

The reservoirs may be presented one after another to the identifier device, so that the substances are placed therein in a given order.

Each terminal may, for example, include a system that is arranged to provide indicator lighting guiding the filling of the reservoirs. In a variant, the reservoirs are filled with the help of a rotary reservoir carrier.

By way of example, the terminal is arranged to identify automatically the substances placed in the reservoirs.

The identification system may also be arranged to enable the quantity of at least one substance in the reservoir that receives it to be evaluated. The identification system may include, for example, means for performing an optical measurement, a conductivity measurement, a pressure measurement, or a gravimetric measurement.

In a variant, the identification system includes one or more chemical receivers, e.g. located above the substances and enabling the presence of the substances to be verified by an analytical method.

The identification system may for example be arranged to inform the user of the terminal that certain reservoirs are empty or full.

### Calculating Quantities

Each terminal may operate in a plurality of reproduction modes, each reproduction mode being associated with a predefined quantity of formulation to be reproduced. The user interface of the terminal is arranged for example to enable the user to select at least one reproduction mode.

By way of example, in a “trial” reproduction mode, a very small quantity of scent is dispensed, e.g. lying in the range 0.01 milligrams (mg) to 10 mg. Such a mode of reproduction is used for example for reproducing a formulation that enables a test scent to be dispensed.

In a “for use” reproduction mode, a quantity lying in the range 10 mg to 2 grams (g) is dispensed, for example.

Finally, in a “fabrication” reproduction mode, a quantity lying for example in the range 2 g to 200 g is dispensed.

The “for use” reproduction mode is adapted for example to reproducing a formulation that results from collaboration between the user of the terminal and the expert associated with the server.

The “fabrication” reproduction mode serves for example to dispense a formulation that is approved by the expert and the user, and that the user desires to use for several days, for example.

The user interface may enable the user to indicate whether the formulation should be reproduced all at once or on several occasions. For example, in the “for use” reproduction mode, the user may act on the user interface of the terminal so that reproduction is performed four times, each comprising 0.05 g of scent, each being dispensed and placed on a different one of four portions of the body.

Similarly, the reproduction of a formulation in a “trial” reproduction mode may be performed several times over, with 1 mg to 10 mg of scent being prepared each time.

Exemplary embodiments of the invention may also enable the user of the terminal to send a request for reproducing a formulation in accordance with one of the reproduction modes of the server and/or to a second terminal and may specify in the request the address to which the scent as dispensed in that way is to be sent, e.g. by a postal operator.

### Dispenser Means

The means for dispensing substances are configured for example to present a flow rate lying in the range 1 milligrams per second (mg/s) to 10 grams per second (g/s), and in particular in the range 10 mg/s to 1 g/s.

By way of example, the dispenser means may be similar to those used in microfluidics, for example, and in particular electrostatic microfluidics, and may enable the scent to be dispensed in the form of a drop or a jet of liquid.

The dispenser means of the invention may include controllable hoses and valves for dispensing small volumes, at rates lying in the range a few microliters per second or even less to a few milliliters per second.

The dispenser means of the invention may be made at least in part, and in particular entirely, out of plastics material, ceramic, or metal. They may also be covered at least in part in a material like as a non-stick material, such as a fluorinated coating, or silica.

The dispenser means may be obtained by molding, etching, or assembly.

The valves may be controlled mechanically, by applying pressure, or by pulling on strings, electromechanically, magnetically, thermally using the property of materials to expand when heated, thereby selectively closing off the passage for a substance.

In a variant, the dispenser means of the invention may include valves based on piezoelectric type materials or on actuators based on a conductive polymer or a dielectric elastomer. Valves may be actuated, for example, in electrical manner, e.g. by using low voltages, in particular voltages of less than 10 volts (V).

In a particular implementation of the invention, the dispenser means do not have any valves, with movement of the substance contained in a reservoir being triggered by the user exerting action, e.g. by pressing on a wall of the reservoir.

The force that enables the substance contained in the reservoir to move when the valve is open may correspond to continuous pressure being applied to all of the reservoirs of the terminal or to pressure being applied solely at the moment the reservoir is actuated.

By way of example, the dispenser means may include one or more pistons, in particular a piston that is moved by an electric force.

In a variant, the dispenser means include one or more pumps or bladed wheels or small propellers for exerting a force on the reservoir that enables the substance to be moved.

In another variant, the dispenser means include means for heating the reservoir, thus enabling the walls of reservoirs to be expanded, or enabling the substance(s) or gas to penetrate into the reservoir.

In another variant, the dispenser means include means arranged to use a pneumatic effect to generate the force that enables the substance contained in the reservoir to be moved. By way of example, the reservoir includes an inlet for air, for example, or for a liquefied gas that is expanded.

In another variant, the dispenser means include one or more electric motors, e.g. a stepper motor for generating the force that enables the substance to be moved, or assisting in moving the substance.

The force enabling the substance to be moved may be applied directly to the substance, or indirectly, e.g. being applied to a flexible wall.

The fluid(s) set into movement by the dispenser means may be dispensed towards an outlet of the terminal where mixing takes place. In a variant, the dispenser means include one or more mixing chambers and the substance(s) are directed to said mixing chamber(s).

The dispenser means may also include means for generating at least one of the following actions in the mixing chamber(s): heating; cooling; pressurization; depressurization; stirring; or indeed inflow of some other substance.

The dispenser means may also include a stirrer unit, e.g. a feed, baffles, or a movable stirrer member that is driven to move.

The dispenser means may also include means for rinsing the pipes and/or the mixing chamber(s). By way of example, such means enable a liquid, a gas whether liquefied or under pressure, or a brush to pass, for example.

In addition to the reservoirs containing substances for reproducing one or more formulations and as described above, the terminal may also include at least one reservoir 13 for a rinsing fluid, shown in FIG. 2, and at least one reservoir for a pressurizing fluid.

Similar to the above description relating to reservoirs for substances of the formulation, the first terminal may also be configured to enable the quantity of rinsing fluid or the quantity of pressurization fluid that remains in the corresponding reservoir to be quantified.

The dispenser means may also include at least one waiting chamber together with one or more valves enabling a first intermediate scent obtained by reproducing a first formulation to be transferred to and stored in the waiting chamber, thus making it possible, for example, to prepare a scent by mixing the first scent stored in the waiting chamber with a second intermediate scent obtained by reproducing a second formulation and prepared after the first formulation was reproduced.

By way of example, the user interface is arranged to enable the user of the terminal to actuate the various elements of the above-described dispenser means. The interface is advantageously adapted to the embodiment selected for making the dispenser means. For example, the user interface is electronic and incorporates self-monitoring systems associated with power stages of the first terminal.

The terminal may include a plurality of distinct dispenser means, e.g. dispenser means specific to each reproduction mode.

The terminal may comprise different portions, e.g. a main body having the user interface and the network interface, together with one or more auxiliary bodies including all or part of the dispenser means, the auxiliary body(ies) being detachable from the main body, for example.

By way of example, the main body is configured to select one or the other of the dispenser means as a function of the quantity of scent that is to be prepared. By way of example, the main body may also be configured to enable the user to confirm a choice of formulation for reproduction and/or a quantity of scent to be prepared.

The terminal may include one or more scent outlets.

By way of example, one scent outlet may be associated with a single one of the dispenser means. In a variant, at least two dispenser means may share a common scent outlet.

Prior to reproducing the formulation, the main body may inform the user of the scent outlet that is to be used.

The scent outlet may include a dispenser nozzle, and where appropriate, applicator means, such as a felt or strip of paper, which may be cleanable or purgeable for example.

The scent outlet may also include an active discharge system, using pressure or suction.

The scent outlet may also include a buffer arranged to enable the prepared scent to be kept available to the user. By way of example, the buffer may be a foam selected from materials that are flexible and non-absorbent, e.g. a polyurethane foam. The scent outlet may include a sintered buffer.

After the formulation has been reproduced, the scent may for example be delivered to the buffer and the user may press the buffer against a finger or some other part of the body in order to recover the stored scent. When the buffer is made of a non-absorbent material, a few presses suffice to enable the user to recover all of the scent.

The scent outlet may be fitted to receive bottles, or systems adapted to olfaction such as a breathing mask.

The scent outlet may also be connected to an air circulation system such as a heater or an air conditioner.

In a variant, the outlet may be connected to an air circulation system opening out into pieces of furniture or particular enclosures such as a cupboard, a car, or a room.

The above-described dispenser means may be incorporated in a terminal or may be associated with the server.

The scent outlet may be provided, where appropriate, with spray means, e.g. piezoelectric spray means.

The dispenser means may also act by evaporation.

### Formulation Reproduction

By means of the invention, the terminal is capable of reproducing formulations that enable a test scent to be dispensed.

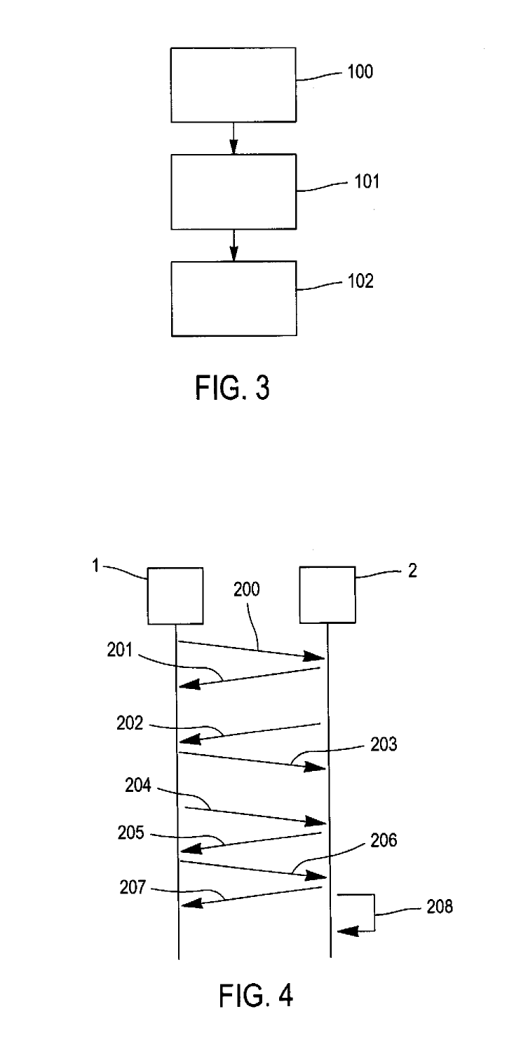

With reference to FIG. 3, there follows a description of a first example of reproduction of such a formulation by a terminal.

The reproduction may be performed in the following three steps.

In a step 100, quantities of each substance of the formulation to be reproduced are adjusted and the quantity of scent to be dispensed is also adjusted entirely automatically by the terminal executing instructions relating to adjustment and transmitted by the server.

In a variant, the user may adjust the volume or the weight of each substance on the basis of indications provided by the server.

In the example shown, it is assumed that all of the substances listed in the formulation for enabling the test scent to be dispensed are available at the terminal.

In step 101, the dispenser means of the terminal are actuated so as to dispense the substances with the quantities determined in step 100. This actuation may be performed at the initiative of the user of the terminal, or automatically.

Finally, in step 102, the distributed substances are mixed so as to obtain the scent of the formulation and the scent is recovered at a scent outlet from the terminal.

Optionally, during a step that is not shown and that may take place before or after step 102, operations of dilution with a solvent or a gas, or operations of concentration, e.g. by evaporation, may be performed on the scent or on one or more of the substances.

Other operations, such as UV irradiation, for example for sterilization purposes or to activate the actions, or indeed heating, may be performed during reproduction. These operations may be performed on the substances, on the result of mixing them, and/or on various portions of the first terminal, such as its pipes, mixing chambers, valves, . . . . As explained below, when the terminal does not contain all of the substances for reproducing the formulation, the terminal may inform the user and may optionally request delivery of the missing substances.

### Operation in Slave Mode

With reference to FIG. 4, there follows a description of a first example of an application of the invention to reproducing a formulation that enables a scent to be dispensed in slave mode by a terminal 1.

The terminal 1 may automatically send a request 200 to the server 2, the request including information indicating that a predefined condition as stored in the memory associated with the terminal is satisfied. The request may include data relating to the odorous substances available at the terminal 1 or that may optionally be placed in the terminal. The predefined condition may for example be the lapse of a certain length of time since the last time the terminal 1 receives a formulation.

The server 2 sends an acknowledge message 201 to the terminal 1, confirming that it has received the request 200.

Thereafter the server 2 analyses the request 200.

Once this analysis has been done, the server establishes that a given formulation can be reproduced by the terminal, possibly after replacing odorous substances listed in the formulation determined by the server and not available at the terminal with odorous substances that are available at the terminal and selected with the help of a correspondence table.

The server then sends the determined formulation to the terminal 1, the formulation being downloaded by the terminal at 202, for example.

Where appropriate, a multimedia content for delivery by the terminal is also downloaded at 202.

The terminal 1 sends an acknowledge message 203.

The user of the terminal 1 then selects the moment at which the user desires the received formulation to be reproduced by performing steps 100 to 102 as described with reference to FIG. 3.

Where appropriate, the user may then send a request 204 to the server 2, the request ordering a packaged product associated with the scent that has been dispensed or that is to be dispensed. The server 2 then sends an acknowledge message 205 to the terminal 1.

In the example described, the terminal 1 sends a request 206 to the server 2 to inform the server that the test scent has been dispensed. The server 2 sends an acknowledge message 207 to the terminal 1 and, at 208, credits or debits an account associated with the user of the terminal.

The server 2 may for example subsequently perform statistical processing.

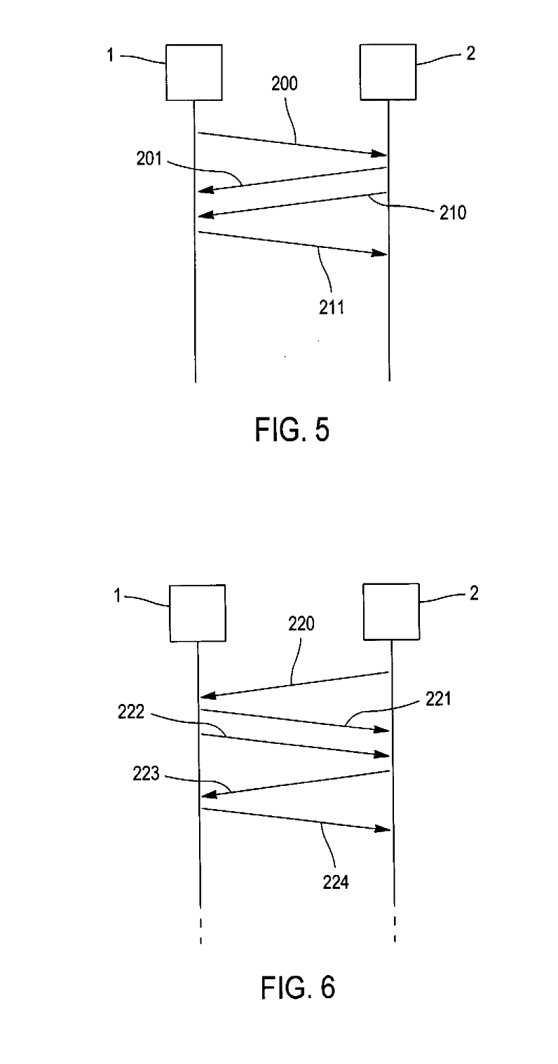

FIG. 5 shows another example of an application of the invention to the terminal 1 reproducing a formulation in slave mode.

In this example, at the end of the analysis of the request 200 by the server, the server establishes that the determined formulation cannot be reproduced by the terminal.

The server then sends a request 210 to the terminal 1 informing the user of the terminal of the substance(s) not available at the terminal for reproducing the formulation that would enable the test scent to be dispensed.

The terminal 1 then sends an acknowledge message 211 to the server 2.

FIG. 6 shows another example of an application of the invention to the terminal 1 reproducing a formulation in slave mode.

In this example, the server 2 acts on its initiative to send a request 220 to the terminal 1 in order to establish whether one or more predefined conditions stored in the database are satisfied. The terminal 1 sends an acknowledge message 221 and then, after searching to see whether the predefined condition(s) is/are satisfied sends a request 222 to the server 2.

When the predefined condition(s) is/are satisfied, the terminal 1 sends, to the server 2, a request 223 similar to the request 200 of FIGS. 4 and 5.

The terminal 1 then sends an acknowledge message 224 to the server 2.

The requests subsequently exchanged between the terminal 1 and the server 2 may be similar to those of the example of FIG. 4 or to those of the example of FIG. 5.

In other variants that are not shown, the predefined condition is verified at the initiative of the server, which is arranged to access the memory in which the user preferences are stored. The server may for example compare the formulations of scents previously purchased by the user of the terminal with the formulations stored in the server and it may then propose sending them to the terminal.

In another variant, the terminal 1 includes a positioning system of the GPS type or as used by mobile telephones and the predefined condition relates to a threshold distance between the server and the terminal. For example, the server may be integrated in a base situated in a scent shop, and the predefined condition is satisfied when the distance between the terminal and the base is less than the threshold distance.

Reproduction in slave mode serves for example to offer a scent in the context of a commercial offer.

In a variant, this reproduction in slave mode is performed in the context of an exercise.

When a terminal is configured to operate in slave mode, it may enable the user to decide to reproduce or not reproduce a formulation and to determine the instant at which the reproduction is to take place.

In the slave mode of operation, the user of the terminal cannot edit or nuance, where appropriate, the formulation(s) received with the help of the above-described nuance reservoirs.

### Other Mode of Operation of the Terminal

In a variant, the terminal is configured to enable the user to edit a received formulation.

For example, the formulation is displayed on the screen of the user interface and the user may act on keys of the user interface to modify the contents of certain substances. For example, the user may modify the proportions of simple substances contained in the nuance reservoirs, or introduce into the formulation new simple substances contained in the nuance reservoirs.

The formulation may also be printed by the terminal.

### Hidden Formulation

By way of example, hidden formulations are stored in the memory associated with the terminal and are accessible to the user only after the terminal has received authorization, e.g. in the form of a code, e.g. transmitted by the server.

Comparison Between the Formulation and the Substances Available. Correspondence Tables for Handling Missing Substances

As described above, the terminal may send data to the server identifying the substances at the terminal, and the server may be configured, on analyzing that data, to compare the odorous substances listed in the predefined formulation that the server is proposing to send to the terminal with the odorous substances identified as being available at the terminal.

In a first server configuration example, the server determines one or more formulations on the basis solely of the substances identified as being available at the terminal.

In a second server configuration example, the server determines one or more formulations on the basis of the substances identified as being available at the terminal, and of one or more substances that are not identified as being available at the terminal, where appropriate.

Under such circumstances, the server may interrupt its analysis and send a warning message to the terminal indicating that certain substances are absent.

In a variant, the database includes a correspondence table between various odorous substances as a function of physicochemical properties common to those substances, and the server determines at least one formulation by comparing the substances available at the terminal with the substances of the formulation to be reproduced, and uses the correspondence table to replace the or each substance in the said formulation that is identified as being not available at the terminal with a substance that is identified as being available at the terminal.

In this example, the invention makes it possible to approximate the final nuance in spite of one or more substances being unavailable or being available in small quantity only.

The correspondence table may operate on broad classes of odor, and on the basis of proximity between odors, it may propose other substances, and possibly correcting the quantities of other substances, where necessary. By way of example, if vanillin is missing, the correspondence table indicates that ethyl vanillin can be used instead. The correspondence table may also specify that ethyl vanillin needs to be used in smaller quantities than vanillin, since it is stronger. The correspondence table may also include information indicating that the quantities of other substances need to be recalculated.

Each odorous substance in the table of correspondences may be associated for example with a list of similar substances, classified by degree of proximity with said odorous substance. The degrees of proximity between odorous substances may be quantified by perfumery experts, for example.

By way of example, a substance A may be associated with six odorous substances: a substance B that is 90% similar to the substance A; a substance C that is 80% similar to the substance A; a substance D that is 70% similar to the substance A; a substance E that is 60% similar to the substance A; and a substance F that is 50% similar to the substance A.

When the server uses the correspondence table to determine whether at least one of the odorous substances listed in the optimized formulation and not available at the terminal may be replaced by at least one other odorous substance that is available at the terminal, it may replace the non-available substance with a substance from the list that is available at the terminal and that is selected by decreasing degree of proximity.

If none of the substances available at the terminal appears in the list of substances associated with the substance listed in the formulation and not available at the terminal, then the server may propose sending the missing odorous substance to the terminal or taking advice from an internal expert in order to find a suitable substance.

In a variant, the database does not have a correspondence table and the terminal is configured to search to find out whether a mixture of substances contained in a reservoir includes the substance(s) identified as being unavailable, or whether the substance(s) is/are contained in reservoirs but at a different concentration.

In another variant, when the server is not fully automatic, the substance(s) of the formulation to be obtained and not available at the terminal is/are replaced by the expert relying on experience.

The substance(s) identified as being unavailable may also be added to the terminal. For example, the server may suggest to the terminal that the substance(s) identified as being unavailable should be loaded into the terminal, or in a variant one or more similar substances should be loaded, and it may specify which reservoirs should be emptied to receive the substance(s) to be introduced. The substances corresponding to the reservoirs for emptying may be selected for example amongst those that are not listed in the formulation, or indeed from amongst those that are the least used.

When the missing substance(s) is/are not immediately available, the server may send an order for the missing substance(s) to a center for shipping substances.

The invention is not limited to the server comparing substances listed in the formulation determined by the server and enabling a test scent to be dispensed using the substances available at the terminal.

Such a comparison may also be performed by the terminal while the terminal is reproducing a formulation. Under such circumstances, the memory associated with the terminal may include, for example, a correspondence table as described above.

Programs for Preparing Formulations for Becoming Familiar with Odors and for Education in Olfaction

In an implementation of the invention, the memory associated with each terminal includes one or more programs for starting the reproduction of one or more formulations. The programs may be implanted in the terminal during manufacture or they may be loaded subsequently.

These programs enable formulations to be reproduced, where some may be no more than very simple mixtures or even pure substances. Independently of dispensing a test scent, these programs may enable formulations to be reproduced, e.g. to discover the possibilities of the terminal, the possibilities of some particular substance, or of a palette of substances, or indeed to educate one or more users about the world of scents. The programs may also enable formulations to be reproduced or formulations to be generated on the basis of formulations that are also defined in order to access the tastes of the user of the terminal in olfactive matters.

These programs may also enable the user of a terminal and the expert associated with the server to agree on a common language for describing the scent obtained by reproducing a formulation. A description may be defined on the basis of a same reproduced odor, with the description being stored in the memory associated with the terminal and/or in the database associated with the server, for example.

The programs may be accompanied by representation, e.g. a description of substances listed in the formulation and of their quantities. The representation may also include images, text, music, film clips, and/or questionnaires.

By way of example, the programs may appear in the form of games.

Such programs enable the invention to be implemented in playful manner, which can increase the attraction of the invention to a user of the terminal and thus make it easier to educate the user.

In an implementation of the invention, a terminal and the server may be used in the care and therapy of adults or children, e.g. suffering from a loss of olfactory sensation, amnesia, or indeed autism, for example.

### Formulation Discovery Program

Independently of dispensing a test scent, the programs stored in the memory associated with the terminal and/or in the database associated with the server may also include programs that, on being executed, present novel products, or existing products by reproducing formulations.

By way of example, programs may present products that are being manufactured.

Programs may also present products by setting out the various elements of a range.

They may also present products by resolving the various notes of the product.

### Systems for Inhibiting and Activating Programs, Formulations, and Substances

Each terminal may include systems for inhibiting the programs described above, and where appropriate representations and questionnaires that might be associated therewith. By way of example, such inhibitor systems may serve to prevent programs being launched either temporarily or permanently.

By way of example, program activation may be possible after the terminal has received an order sent by the server.

In a variant, the inhibitor system is arranged to prevent one or more programs being activated until a predefined date or for a predetermined duration. By way of example, the predetermined duration may correspond to a duration of prior operation of the terminal.

In another variant, the inhibitor system is arranged to be deactivated after a given code has been entered via the user interface of the terminal, or indeed after the terminal has been connected to a given computer key.

In order to enable a scent to be distributed, exemplary embodiments of the invention may thus prevent it from being discovered until a given date, with the formulation being hidden, as described above, in the memory associated with the terminal prior to the terminal receiving an authorization in the form of a code.

By way of example, the inhibitor system is configured to allow a formulation to be reproduced only under predefined conditions or only for a predetermined number of times.

### Transmission of Formulations and Programs, Automatic Transmission of Formulations

As described above, the terminal may receive formulations sent by the server.

Formulations, and more generally information may be transmitted between the terminal, the server, and where appropriate other terminals, on the basis of wired or wireless connections.

When the terminal and the server are remote from each other, the connection may be constituted for example by a telephone connection or the Internet, with the terminal and the server including the usual interface modules such as modems, routers, etc. By way of example, the interface modules enable WiFi or WiMAX connections to be established.

When the terminal and the server are close together, the connection may involve a wired connection using a cable, an infrared connection, or a radio connection, e.g. a Bluetooth or a ZigBee connection.

In another variant, communication between the terminal and the server when located close to each other may take place by information being entered manually by terminals users or the expert associated with the server, or electronically by means of a memory, such as a universal serial bus (USB) key for example, or by being sent via a relay appliance, such as for example: a mobile telephone or a digital personal assistant.

Other communications means are also possible, such as the user of the terminal or the expert associated with the server receiving a formulation verbally, by the post, by a fax, or by email, and introducing the formulation into the memory of the terminal or the server via the user interface.

### Taking Account of Information for Assisting in the Personalization of Mixtures

### Programs for Identifying Preferences

By way of example, the server may be configured to take account of personal information stored in the user preferences associated with the user of the terminal, such as the user's age, taste, or even country, in order to devise formulations that enable a test scent to be dispensed or to select such formulations as a function of the user.

The user interface of the terminal and/of the server may, for example, be configured to enable the user or the expert to input the data mentioned in the preceding paragraph.

Independently of dispensing test scents, each terminal may also include a comparison system configured to evaluate whether a selection of preferred and/or detested odors made by a user of the terminal is similar to the selection made by some other person, a group of people, or a symbolic group of people.

Each terminal and/or server may also be configured to analyze how formulations vary, so as to extract the preferred modifications made by users.

### Interface for Managing Sent and Received Formulations

Each terminal may include an interface for managing sent and received information.

The terminal interface for managing sent and received formulations includes for example an audible and/or visual indication that a formulation enabling a test scent to be dispensed has been received from the server.

In a variant, the interface for managing sent and received formulations is connected to a computer and/or telephone network and sends a message, e.g. an SMS or an email to the user of the terminal when a formulation for dispensing a test scent has been received.

The interface for managing sent and received formulations may, for example, be configured to store the received formulations in a folder of the memory associated with the terminal and to classify them by type of substance, type of formulation, or by sender.

### EXAMPLES

Various implementations of the invention are described below. In the example described, the communications standard comprises three sections.

The first section is a table having 4096 rows, each row corresponding to a predefined substance. Each row contains a number lying in the range 0 to 255.

The second section is a set of commentaries, made up of 4096 ASCII characters.

The third section comprises information about the identifier of the terminal user, the time and date a formulation is sent, a number identifying the formulation, and a table of eight rows corresponding to the substances available at the terminal.

In the example described, the standard does not contain information about the nature and the description of the substances.

Still in this example, the terminal and the server share a common conversion table, enabling each substance to be translated into an olfactory descriptor.

### Examples Involving Designing, Preparing, and Exchanging Formulations and Experiences

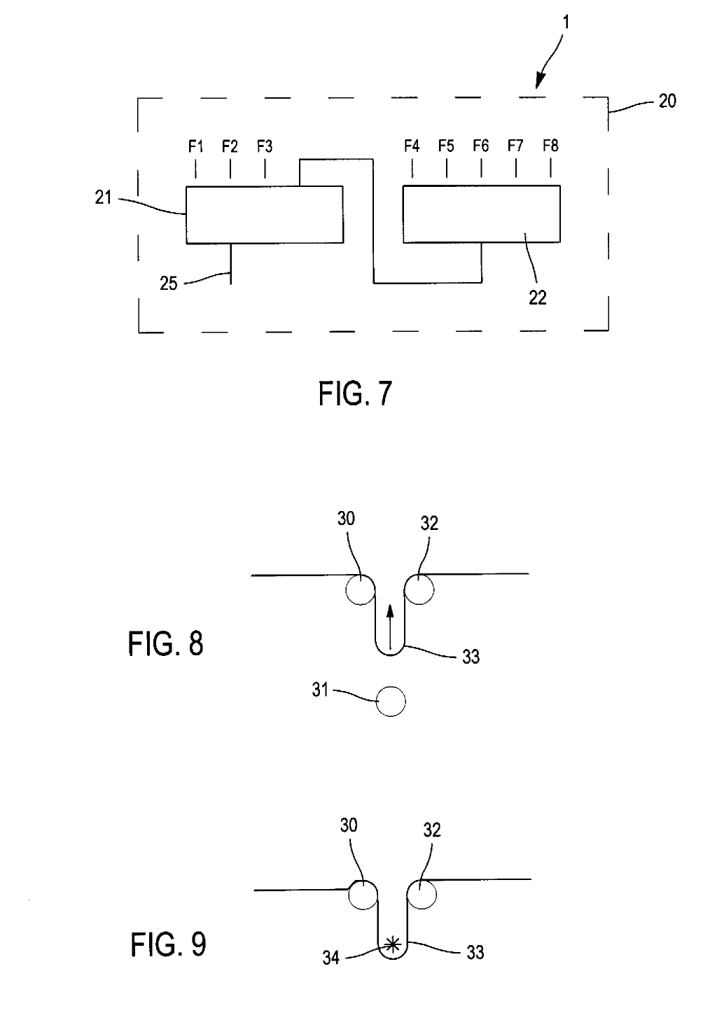

There follows a description of a first embodiment of a terminal of the invention arranged to operate in the “for use” reproduction mode described above. The terminal is shown in highly diagrammatic and fragmentary manner in FIG. 7.

In this example, a common housing 20 contains:
an ultracompact personal computer (PC) of the PC 104 type sold by Advantech® under the reference PCM4170;
a 32-bit input/output card adapted to the PC format;
an eight-relay card adapted to the PC format;
two microfluidic units 21 and 22 sold by the supplier Microliquid® under the reference 3020PCB, having six fluid flow inlets and electrical connectors;
three main 10 milliliter (mL) reservoirs of flexible plastic F1, F2, and F3 corresponding to complex odorous substances; and
five nuance 2 mL reservoirs of flexible plastic F4 to F8 corresponding to simple odorous substances.

The reservoirs and the microfluidic units are connected together as shown in FIG. 7.

The outlet pipe 23 opens out onto a pad (not shown) of expanded polyurethane foam.

The eight reservoirs F1 to F8 are subjected to mechanical pressure by a plate system compressed by three steel springs.

In second exemplary embodiments of a terminal arranged to operate in the “for use” reproduction mode, the terminal does not have microfluidic units.

In these exemplary embodiments, the eight reservoirs F1 to F8 are connected to a mixing chamber by flexible hoses.

The hoses are parallel. Halfway along they follow a bend that is determined by three pulleys 30, 31, and 32, as shown in FIG. 8.

As can be seen, the shaft of the middle pulley 31 is mounted on a rail and can be moved vertically by a stepper motor (not shown). When the pulley moves upwards, the hose 33 is less flattened and allows the fluid to pass.

In these exemplary embodiments, the PC has eight stepper motors controlled using an eight-relay card so as to allow one or more fluids to pass by acting on each of the eight motors.

As shown in FIG. 9, in a variant of these second exemplary embodiments, the middle pulley 31 is replaced by a toothed gearwheel 34.

The shaft of this gearwheel is mounted to be rotated by a stepper motor so as to cause the liquid to flow.

The PC includes a screen, a keyboard, and a mouse.

There follows a description of an example of a program for the PC of a terminal that is arranged to operate in the “for use” reproduction mode.

The PC 104 has the following programs:
a starting interface program referred to as “I-Start” which asks the user whether the locations of the substances have been modified or whether the substances have been changed. If so, the user fills in a questionnaire specifying, for each of the eight locations of the reservoirs, the references of the substances that are placed therein in the form of numbers lying in the range 0 to 4095. This table is referred to as the “Substance-location” table;
an interface program “I-User” displaying three points on the screen representing the three complex odorous substances in the reservoirs F1 to F3, and five vectors starting from the three points, symbolizing the five nuances made possible with the help of the five simple odorous substances in the reservoirs F4 to F8.

By way of example, this program causes the name of each of the substances to be displayed on the screen on the basis of conversion means common to the terminal, to the server, and where appropriate to another terminal, the conversion means associating the references 0 to 4095 with the descriptions of each of the substances.

If the memory associated with the terminal contains formulations, whether formulations that have been received or formulations that have been designed, the most recent formulation to have been recorded is displayed on the screen in the form of a starting point together with the vectors added thereto, and corresponding to the possible additions of nuances. Where appropriate, the screen thus makes it possible to display the other formulations, where necessary.

By means of the interface of the invention, the user can add or remove nuance vectors and/or select one of the three complex starting substances. The complex starting scent is selected, for example, by entering the value 0 or the value 100 into each of three sub-memories each associated with a respective one of the complex starting substances. One or more nuance vectors may also be selected, and for each nuance vector the magnitude thereof may be a multiple of 5%. These vectors are associated with five sub-memories referenced V1 to V5, with it being possible to enter a value lying in the range 0 to 100 in each of said sub-memories.

The interface may also enable the user to input comments in the form of a string of up to 4096 alphanumeric characters.

The PC may further comprise:
a formulation calculation program “Formulation-calculation”, making use on each occasion of a table made up of 4086 rows corresponding to each of the 4096 possible substances. This table may be named “Formulation”. The program is arranged to enter into each of the rows of the table, in the place of the reference for the substance, the values D1, D2, D3, V1, V2, V3, V4, and V5, with each value being reduced by being divided by the value (D1+D2+D3+V1+V2+V3+V4+V5);
a formulation transmission program referred to as “Formulation-transmission”, whereby the terminal uses an asymmetric digital subscriber line (ADSL) modem incorporated in the PC to send the formulation file, the commentary file, and the “user” file. The user file may be of a size of about 4096 bytes and may contain the following data: the user code, the date and time of sending, and the “substance-location” table. The “Formulation-transmission” program also enables the terminal to receive formulation files, to generate a warning for the user by showing the date and time the formulation was received on the screen, and also showing formulation expressed by the list of its substances. This program also makes it possible to analyze the reproduction of the formulation, by running the program “I-Preparation” after obtaining agreement from the user;
an interface program for preparing reproduction referred to as “I-Preparation”, whereby the interface verifies that the substances of the formulation are compatible with the substances available at the terminal, and warns the user if a substance is missing, and requests the quantity of scent to be dispensed and authorization to activate the reproduction program;
a formulation reproduction program referred to as “Formulation-reproduction”, whereby the terminal converts the formulation into substances that are to be activated by making an eight-row file referred to as “substance-activation”, corresponding to each of the substances and allocating thereto the value from the “formulation” table as multiplied by the previously-input quantity of scent to be prepared. The program also actuates the valves of the dispenser means for a given length of time, corresponding, for each valve for each substance, to the value in the “substance-activation” table, e.g. multiplied by 0.01. The formulation reproduction program may also compare the quantity of scent that is to be dispensed with a threshold of 2 g, for example, and if the threshold is exceeded, it may prevent reproduction of the formulation by the terminal since the terminal is in “for use” reproduction mode. The server may then send a request to reproduce the formulation to a second terminal that is in “manufacturing reproduction” mode.

In the example described, the reservoirs F1 to F8 in each of the terminals are filled with an odorous substance comprising 25% active material and 75% ethanol:
F1 corresponds to a complex scent substance for a conventional man, Aqua di Gio™ from Giorgio Armani®;
F2 corresponds to a complex scent substance for a sportive man Polo Blue™ from Ralph Lauren®;
F3 corresponds to a complex scent substance for a young man Armani Code™ from Giorgio Armani®;
F4 corresponds to a simple substance with a woody dominant, e.g. coming from sandalwood, cedar wood, or vetiver;
F5 corresponds to a simple substance with a simple dominant, such as an essential oil of lemon, an essential oil of bergamot, an essential oil of orange flowers (neroli);
F6 corresponds to a simple substance having a musk dominant, such as white musk, galaxolide;
F7 corresponds to a simple substance having a marine dominant, such as calone; and
F8 corresponds to a simple substance having a gourmand dominant, such as ethylmaltol or ethyl vanillin.

In the example described, the server is associated with a person who is a professional in scent creation. The server has reservoirs containing the same substances as described above.

In this example, the server also has a stock of two hundred substances forming quite a large collection of odorous possibilities in the field of odors for alcohol-based scents.

In the example described, the server and the two terminals are connected together via a telephone network connection capable of transmission at 2 megabits per second (Mb/s). The two users and the expert begin by running the program “I-Start”.

There follows a description of several examples of how the invention may be used.

### Example 1

### Dispensing a Test Scent

The server transmits a formulation enabling a test scent to be dispensed at the terminal, by using the above-described “Formulation-transmission” program, e.g. after verifying that at least one of the above-mentioned predefined conditions is satisfied, e.g. a condition relating to the location of the terminal relative to a scent shop or an upcoming birthday or other celebration date for the user of the terminal or of a relative.

Warned by the reception of the formulation, the user of the terminal decides to dispense the test scent by reproducing the received formulation, and deciding to prepare a quantity of 5 mg.

The terminal then sends a request to the server informing it of the dispensing of the test scent and credits an account associated with the user in order to recompense it.

### Example 2

### Dispensing a Test Scent with the User Having the Option of Adding a Nuance

The server transmits a formulation enabling a test scent to be dispensed at the terminal, e.g. in similar manner to Example 1.

The received formulation is displayed on the user interface of the terminal and the user decides to add a nuance, where appropriate after reproducing the formulation as received, for the purpose of increasing or decreasing the content of one or more of the simple substances contained in the nuance reservoirs and/or by introducing additional simple substances into the formulation.

The user then acts on the terminal to reproduce the nuanced formulation.

### Example 3

### Dispensing Test Scents with a User Selection Option

The server transmits four formulations enabling test scents to be dispensed by the terminal, e.g. in similar manner to Example 1.

The received formulations are displayed on the user interface of the terminal and the user decides to classify them in order of preference, where appropriate after reproducing the received formulation.

### Example 4

### Dispensing a Test Scent with an Analysis Option

The server transmits a formulation enabling a test scent to be dispensed at the terminal, e.g. in similar manner to Example 1.

The received formulation is displayed on the user interface of the terminal and the user decides to return an opinion, where appropriate after reproducing the received formulation. This may be done by means of a free-form reply or by filling in a predefined form of possible opinions.

### Example 5

### Dispensing a Test Scent as Proposed by a Third Party

A third party makes contact with the server to find out whether a particular scent will make an acceptable gift for an intended recipient. The server then transmits a formulation anonymously that enables a test scent to be dispensed by the terminal of the intended recipient, for example in similar manner to Example 1.

The received formulation is displayed on the user interface of the intended recipient's terminal who decides to give an opinion on the received formulation, where appropriate after reproducing the received formulation. The server makes contact with the third party to forward the opinion of the intended recipient.

The invention is not limited to the examples described above.

In particular, it is possible to combine the characteristics described with reference to different embodiments with one another.

Although in the examples described the formulation corresponding to the test scent for dispensing is sent by a server, the invention does not exclude the formulation being sent by a second terminal, then acting as a server.

The term “comprising a” should be understood as being synonymous with “comprising at least one”.

1. A method of dispensing at least one test scent within a system comprising a server configured to exchange data with a plurality of interactive scent-formulation terminals, each interactive terminal comprising:
a plurality of odorous substances configured to be combined in various proportions to create a plurality of scents;
a dispenser for dispensing one or more of the odorous substances from the terminal;
a network interface enabling the terminal to communicate with the server; and
a user interface;
the method comprising:
automatically sending data from the server to at least one of the plurality of interactive scent formulation terminals, the data describing at least one formulation for dispensing by the terminal as a test scent, while enabling a user of the terminal to select a moment at which the test scent is dispensed.

2. The method according to claim 1, wherein the server automatically sends data describing the same formulation to more than one of the plurality of terminals.

3. The method according to claim 1, further comprising selecting by the user of the terminal is one or more of the odorous substances for dispensing via the user interface.

4. The method according to claim 1, wherein the test scent formulation described in the data sent by the server is determined as a function of user preferences associated with the user of the terminal to which the data is sent.

5. The method according claim 1, wherein the sending of data describing the formulation of the test scent to a terminal is conditional on the server detecting a predefined condition associated with the user of the terminal.

6. The method according claim 5, wherein the predefined condition associated with the user of the terminal relates to the odorous substances available at the terminal, and wherein the server informs the terminal of the substance(s) listed in the formulation for dispensing the test scent that is/are not available at the terminal.

7. The method according to claim 1, wherein the user of the terminal is able to order a packaged product associated with the test scent of formulation described in the data sent by the server.

8. The method according to claim 1, wherein the data describing the formulation of the test scent and sent by the server refer to a formulation stored in a memory associated with the terminal.

9. The method according to claim 1, wherein said stored formulation is hidden, access to the formulation by the user of the terminal being made possible only after receiving an authorization delivered by the server.

10. The method according to claim 1, wherein the server debits or credits an account associated with the terminal when the user of the terminal orders dispensing of the test scent.

11. The method according to claim 1, wherein the user of the terminal receiving the data describing the formulation of the test scent is able to modify the received formulation.

12. The method according to claim 1, wherein the user of the terminal that has received the data describing the formulation of the test scent is able to forward said data to another terminal selected by the user.

13. The method according to claim 12, wherein the user is able to accompany the transmission a transmission of the data describing the formulation of the test scent by a message.

14. The method according to claim 1, wherein the server transmits multimedia data associated with the data describing the formulation of the test scent.

15. The method according to claim 1, wherein the data automatically sent to the at least one terminal by the server describe a formulation that has not been previously reproduced by the terminal.

16. The method according to claim 1, wherein the dispenser means, the user interface and the network interface are incorporated in a single device.

17. The method according to claim 1, wherein the odorous substances are contained in reservoirs capable of being placed in the terminal independently of one another.

18. A server configured to exchange data with a plurality of interactive scent-formulation terminals, each terminal comprising:
a plurality of odorous substances, configured to be combined in various proportions to create a plurality of scents;
a dispenser for dispensing one or more of the odorous substances from the terminal;
a user interface; and
a network interface enabling the terminal to communicate with the server, so as to receive data from the server describing at least one formulation for dispensing as a test scent;
the server being configured to send data automatically to at least one terminal, the data describing at least one formulation for dispensing by the at least one terminal at least one test scent, and to receive from the at least one terminal a message confirming that the test scent has been dispensed.

19. The method according to claim 5, wherein the predefined condition relates to at least one of a location of the terminal, a use of the terminal, a use of computerized payment means, and the plurality of odorous substances available at the terminal of the user.
